(19) United States
(12) Patent Application Publication
(10) Pub. No.: US 2023/0149463 A1
(43) Pub. Date: May 18, 2023

(54) METHOD OF EXPANDING AND GENERATING A POPULATION OF CYTOKINE-INDUCED KILLER CELLS FROM PERIPHERAL BLOOD

(71) Applicant: Cytopeutics Sdn. Bhd., Cyberjaya Selangor (MY)

(72) Inventor: Bernard Sze Piaw Chin, Cyberjaya Selangor (MY)

(21) Appl. No.: 17/974,123

(22) Filed: Oct. 26, 2022

(30) Foreign Application Priority Data
Nov. 17, 2021 (MY) PI 2021006827

(51) Int. Cl. A61K 35/17; C12N 5/0783

(52) U.S. Cl. CPC A61K 35/17; C12N 5/0646; C12N 2501/2302; C12N 2501/2315; C12N 2501/515; C12N 2533/56; C12N 2533/50; C12N 2501/24; C12N 2501/599

(57) ABSTRACT

The present invention relates to a method of expanding and generating a population of cytokine-induced killer (CIK) cells from peripheral blood mononuclear cells comprising steps of a) separating the mononuclear cells from the peripheral blood; b) transferring the separated mononuclear cells into a culture medium; c) adding cytokines into the culture medium to induce expansion and generation of the CIK cells; and d) obtaining the expanded and generated population of CIK cells.

### CROSS-REFERENCE TO RELATED APPLICATIONS

This application claims priority to Malaysian Application No. PI 2021006827, filed on Nov. 17, 2021. The content of the prior application is hereby incorporated herein by reference in its entirety.

### FIELD OF INVENTION

The present invention relates to a method of expanding and generating a population of cytokine-induced killer (CIK) cells from peripheral blood for cellular immunotherapy.

### BACKGROUND ART OF THE INVENTION

Malignancy in cancer has constituted high mortality rate despite medical advancements in diagnosis and treatment. Conventionally, cancer treatments such as surgery, chemotherapy and radiotherapy have been employed to remove or destroy cancer cells. Although having demonstrated to confer benefits to patients who are afflicted by cancer, however, these treatments have also been associated with serious and long term side effects, eventually reducing quality of life. It is understood that chemotherapy and cancer itself can deplete the immune cells which further suppress and weaken the body's ability to fight cancer.

In light of the above, medical experts have begun to look into other alternatives treating cancer. In recent years, adoptive cell therapy which is also known as cellular immunotherapy, has emerged as a potential adjuvant approach in cancer treatment by utilising the body's own immune system to fight cancer. Adoptive immunotherapeutic cells such as cytokine-induced killer (CIK) cells offer a promising potential in cellular immunotherapy.

CIK cells are a group of immune cells comprising T-lymphocytes, natural killer (NK) cells and natural killer-T (NKT) cells which can be generated from peripheral blood mononuclear cells (PBMC). CIK cells are highly proliferative and can effectively kill tumour cells without specific tumour antigen, as they do not require interaction with T-cell receptor (TCR) and presence of major histocompatibility complex (MHC) molecules. In the CIK cells population, NKT cells are identified by the presence of CD3⁺ and CD56⁺ surface markers, and are primarily responsible for the non-MHC restricted cytolytic activities against tumour cells. It was reported that CD3⁺CD56⁺ NKT cells exhibiting features of both T cells and NK cells possessed superior anti-tumour effect in the treatment of blood and solid cancers including cancers of the lung, stomach, pancreas, liver, colon, breast, prostate and others (Guo & Han, 2015). In addition, treatment with CIK cells has been proven to achieve cancer remission with mild side effects, and to provide immunological boost even in patients with advanced disease stage. The combination of treatment using CIK cells together with conventional therapies also improved disease-free survival and prolong overall survival in cancer patients as compared to standard therapy alone (Jäkel et al., 2014).

One of the major challenges to a successful treatment with application of CIK cells is the isolation and expansion of appropriate effector cells from PBMC. Apheresis, a machine-operated medical procedure for separating specific blood components, is widely used to obtain PBMC for generating CIK cells. However, a large volume of blood is required for the apheresis to obtain sufficient PBMC and most critically-ill cancer patients are unable to tolerate the procedure as it is profoundly arduous and burdensome. Studies have also shown that PBMC isolation rate via apheresis was relatively low and ample amount of cellular debris was observed in the apheresis product, which might increase the risk of adverse effects in patients (Liu et al., 2015). Apart from that, the cost of apheresis is also a concern as it is rather expensive.

Therefore given the potential of cellular immunotherapy for treatment of cancer, there remains a need for a method to utilise a relatively small volume of blood to generate sufficient CIK cells without increasing the burden of cancer patients, thus, motivating inventor of the present invention to develop a novel method of generating a population of CIK cells from peripheral blood mononuclear cells for cellular immunotherapy.

### SUMMARY OF THE INVENTION

In alleviating limitations resulting from the foregoing conventional methods, the present invention provides a method of expanding and generating a population of CIK cells.

More particularly, the present invention relates to a method of expanding and generating a population of CIK cells from peripheral blood mononuclear cells wherein the method comprises steps of a) separating the mononuclear cells from the peripheral blood; b) transferring the separated mononuclear cells into a culture medium; c) adding cytokines into the culture medium to expand and generate the CIK cells; and d) obtaining the expanded and generated population of CIK cells. The population of CIK cells expanded and generated by the present invention includes CD3⁺CD8⁺ T-lymphocytes, CD3⁻CD56⁺ natural killer (NK) cells and CD3⁺CD56⁺ natural killer-T (NKT) cells which will be used in cellular immunotherapy for treatment of cancer.

Other features and advantages of the present invention are readily apparent from the following detailed description with the accompanying drawings, which illustrate, by way of example, but not limiting in scope, the various embodiments of the present invention.

### BRIEF DESCRIPTION OF DRAWINGS

The drawings constitute part of this specification and include an exemplary or preferred embodiment of the present invention, which may be embodied in various forms. It should be understood, however, that the figures disclosed herein are not to be interpreted as restrictive, but rather intended as an illustrative basis for teaching a person having ordinary skill in the art of the present invention.

In the appended figures:

FIG. 1 illustrates median total counts of CIK cells for metastatic cancer patients and for healthy donors during 21 days of culture.

FIG. 2 illustrates median fold increase of total CIK cell counts for cancer patients and for healthy donor against Day 1 culture.

FIG. 3 illustrates representative composition of CIK cells from a cancer patient, consisting of CD3⁺CD8⁺ T cells, CD3⁻CD56⁺ NK cells and CD3⁺CD56⁺ NKT cells. R1: total lymphocytes; R2:CD3⁺CD8⁺ T cells; R3: CD3⁻CD56⁺ NK cells; R4: CD3⁺CD56⁺ NKT cells.

FIG. 4 illustrates in vitro cytotoxic killing activity of (a) NK cells; and (b) CIK cells against MDA-MB-231 breast cancer cell line at an effector to target ratios of 1:1, 5:1 and 10:1. MDA-MB-231 breast cancer cell line added with only RPMI, NK media or CIK media without NK or CIK effector cells were used as normalising control.

FIG. 5 illustrates in vitro cytotoxic killing activity of CIK cells generated from healthy donor and cancer patients against (a) MDA-MB-231 breast cancer cell line; (b) H1975 lung cancer cell line; and (c) HCT15 colorectal cancer cell line, at an effector to target ratio of 1:5, 1:2, 1:1, 5:1, 10:1, 20:1 and 40:1. Cancer cell line without the effector CIK cells were used as normalising controls. Data showed were means±SEM. (*p<0.05, **p<0.01)

FIG. 6 illustrates a collection of images depicting CIK cells and cancer cells at effector to target (E:T) ratio of 1:1 and 20:1, as compared to the control well with cancer cells only at 72-hour post treatment. Red arrows represent floating cancer dead cells; green boxes represent CIK cells; red circles represent cancer cells that were still adhered to the surface of the culture vessel, and the circular suspensions and clumps observed in the well with E:T ratio of 20:1 were the CIK cells. Magnification 100×.

### DETAILED DESCRIPTION OF THE INVENTION

A detailed description of the present invention is described herein. The present invention is directed to a method of expanding and generating a population of CIK cells. More particularly, the present invention relates to a method of expanding and generating a population of CIK cells from peripheral blood mononuclear cells wherein the method comprises steps of a) separating the mononuclear cells from the peripheral blood; b) transferring the separated mononuclear cells into a culture medium; c) adding cytokines into the culture medium to expand and generate the CIK cells; and d) obtaining the expanded and generated population of CIK cells.

In a preferred embodiment of the present invention, the peripheral blood is collected from a subject by way of venipuncture. Typically, density gradient centrifugation of the peripheral blood is carried out to separate the mononuclear cells from the peripheral blood.

Next, the separated mononuclear cells are transferred into a tissue culture flask coated with muromonab CD3 (OKT3) or an extracellular matrix protein, preferably, but not limiting to laminin, collagen, fibronectin or vitronectin, in which the tissue culture flask contains a culture medium mixture of T-lymphocyte culture medium, human autologous plasma and interferon gamma (IFN-γ).

Subsequently, the mononuclear cells are cultured in the culture medium for a duration from 12 to 24 hours for cell proliferation. Fresh culture medium is replaced in every 2-3 days continuously for a period of 14-21 days. Selected groups of cytokines including interleukin-2 (IL-2) and interleukin-15 (IL-15) are added into the culture medium to induce expansion and generation of the population of CIK cells. After 12 to 24 hours, IL-2 and OKT3 are added into the culture medium to further induce the cells. Meanwhile, IL-2 and IL-15 are added into the culture medium on day 4 and day 8 of culture.

In another preferred embodiment of the present invention, the expanded and generated population of CIK cells are obtained in a form of suspension of cells after 14-21 days of culture.

In yet another preferred embodiment of the present invention, the expanded and generated population of CIK cells includes CD3⁺CD8⁺ T-lymphocytes, CD3⁻CD56⁺ NK cells and CD3⁺CD56⁺ NKT cells which will be used in cellular immunotherapy for treatment of cancer.

The following examples are provided to further illustrate the present invention and are not to be interpreted as limiting the scope of the invention. Indeed, the present invention is in no way limited to the specific materials and parameters mentioned. A person having ordinary skill in the art may develop equivalent means without the exercise of an inventive capacity and without departing from the scope of the present invention.

### Example 1: Collection and Preparation of Peripheral Blood Mononuclear Cells

A volume from 60 mL to 80 mL of peripheral venous blood was withdrawn from 9 cancer patients (Male; 6, Female; 3) with mean age of 56 years old, and 2 healthy donors (Male; 2) with mean age of 49 years old, using simple venipuncture technique.

Upon collection of the peripheral blood sample, the mononuclear cells were separated from the peripheral blood by way of density gradient centrifugation at 800×g for 30 minutes without brake and at room temperature.

### Example 2: Expansion and Generation of CIK Cells

Subsequently, the separated mononuclear cells were then transferred into a tissue culture flask containing a culture medium mixture of T-lymphocyte culture medium, 0.5% human autologous plasma and 1000 U/mL interferon gamma IFN-γ and were cultured for a duration from 12 to 24 hours. Prior to placing the culture medium mixture into a tissue culture flask, a mixture containing either OKT3 or laminin and a saline solution, preferably but not limiting to phosphate buffer saline, was used to coat the surface of tissue culture flask for a duration from 2 to 24 hours at a temperature ranging from 4 to 40° C.

Next, the CIK cells were induced with addition of IL-2 in a concentration ranging from 300 U/mL to 1000 U/mL and OKT3 in a concentration ranging from 100 ng/mL to 400 ng/mL into the culture medium for another duration from 12 to 24 hours.

The CIK cells were further induced with addition of IL-2 in a concentration ranging from 300 U/mL to 1000 U/mL in every 2-3 days for a period of 14-21 days and IL-15 in a concentration ranging from 10 ng/mL to 50 ng/mL into the culture medium on day 4 and day 8 of culture.

### Example 3: Analysis of the Expanded and Generated CIK Cells

After 14-21 days of culture, the CIK cells were obtained in a form of suspension of cells. The proportion of CD3⁺CD8⁺ T-lymphocytes, CD3⁻CD56⁺ NK cells and CD3⁺CD56⁺ NKT cells of the expanded and generated population of CIK cells was determined by way of flow cytometry analysis.

### Example 4: Results and Discussion

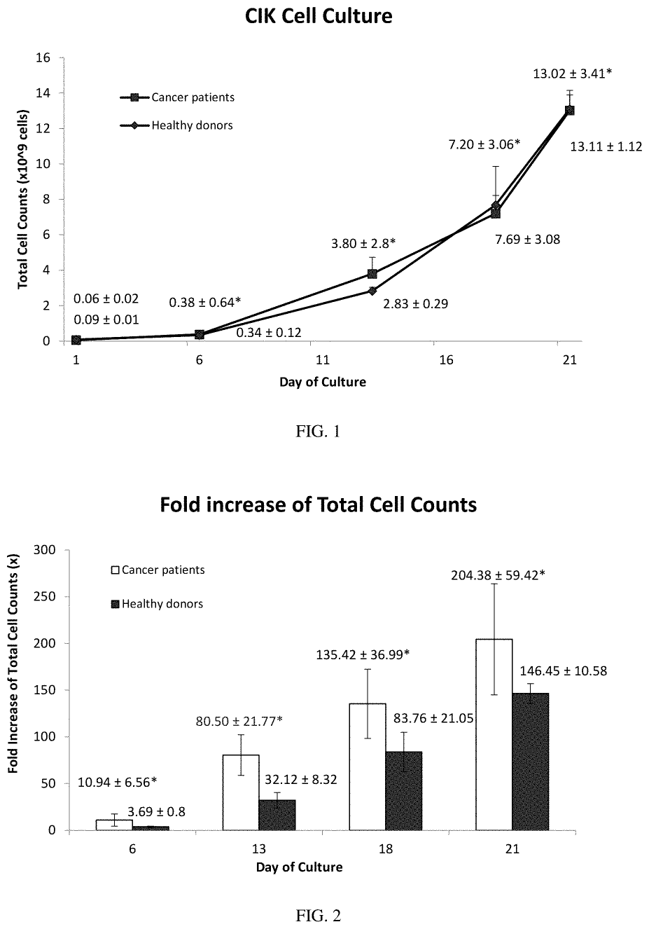

CIK cells were successfully expanded and generated from all 11 blood samples. In cancer patients, the total cell counts were significantly increased from Day 1 to Day 21, with the median expansion number of 0.06±0.02×10⁹ cells vs. 13.02±3.41×10⁹ cells respectively (p<0.01) as shown in FIG. 1, and a median of 204.38±59.42 fold increase as shown in FIG. 2.

Similarly, in healthy donors, the total cell counts were increased from Day 1 to Day 21, with the median expansion number of 0.09±0.01×10⁹ cells vs. 13.11±1.12×10⁹ cells, respectively (FIG. 1), and a median of 146.45±10.58 fold increase (FIG. 2). The comparison cell count data and fold increase between Day 6, Day 13, Day 18 and Day 21 against Day 1 were calculated by using Wilcoxon Signed-Rank Test (p<0.01).

In addition, the median proportion of CD3⁺CD56⁺ NKT cells in cancer patients and healthy donors were 35.14±8.5% and 25.46±3.7% respectively as shown in TABLE 1. The data recorded was shown to have a higher ratio of CD3⁺CD56⁺ cells as compared to CIK population produced using conventional methods.

TABLE 1
Clinical characteristics of subject during baseline assessments, total CIK
cell count and sub-fraction of T-lymphocytes, NKT cells and NK cells
count during 21 days of culture

| Subject | | | | Total CIK Cell Count (×10⁹ ceils) | | | | | T cell count/ | NK Count/ | NKT count/ |
|---|---|---|---|---|---|---|---|---|---|---|---|
| ID | Age | Gender | Diagnosis | Day 0 | Day 6 | Day 13 | Day 13 | Day 21 | CD3⁺CD8⁺ (%) | CD3⁺56⁺ (%} | CD3⁺56⁺ (%) |
| 1 | 70 | M | Metastatic lung cancer | 0.10 | 2.17 | 11.79 | 13.00 | 17.87 | 80.10 | 0.43 | 43.49 |
| 2 | 70 | M | Metastatic Sung cancer | 0.07 | 1.04 | 5.82 | 12.19 | 14.39 | 83.61 | 0.42 | 46.04 |
| 3 | 67 | M | Metastatic. low rectal: cancer | 0.06 | 0.30 | 3.40 | 5.20 | 8.60 | 85.88 | 0.43 | 40.33 |
| 4 | 79 | M | Metastatic pancreatic cancer | 0.07 | 0.84 | 4.62 | 8.88 | 15.47 | 73.38 | 0.04 | 30.41 |
| 5 | 55 | F | Metastatic pancreatic cancer | 0.05 | 0.83 | 4.10 | 8.72 | 8.76 | 62.60 | 0.15 | 29.34 |
| 6 | 60 | M | Metastatic prostate cancer | 0.06 | 0.15 | 3.80 | 5.85 | 13.02 | 82.57 | 0.4 | 35.14 |
| 7 | 60 | M | Metastatic prostate cancer | 0.04 | 0.20 | 3.22 | 7.20 | 13.21 | 84.07 | 0.12 | 22.72 |
| 8 | 42 | F | Ovarian cancer | 0.04 | 0.38 | 3.00 | 4.84 | 8.99 | 69.01 | 0.49 | 43.86 |
| 9 | 42 | F | Ovarian cancer | 0.03 | 0.35 | 2.90 | 5.12 | 9.30 | 69.37 | 0.65 | 26.45 |
| 10 | 50 | M | Healthy donor | 0.10 | 0.43 | 2.62 | 9.87 | 13.90 | 83.89 | 2.09 | 22.84 |
| 11 | 48 | F | Healthy donor | 0.08 | 0.25 | 3.04 | 5.51 | 12.32 | 74.24 | 0.37 | 28.07 |

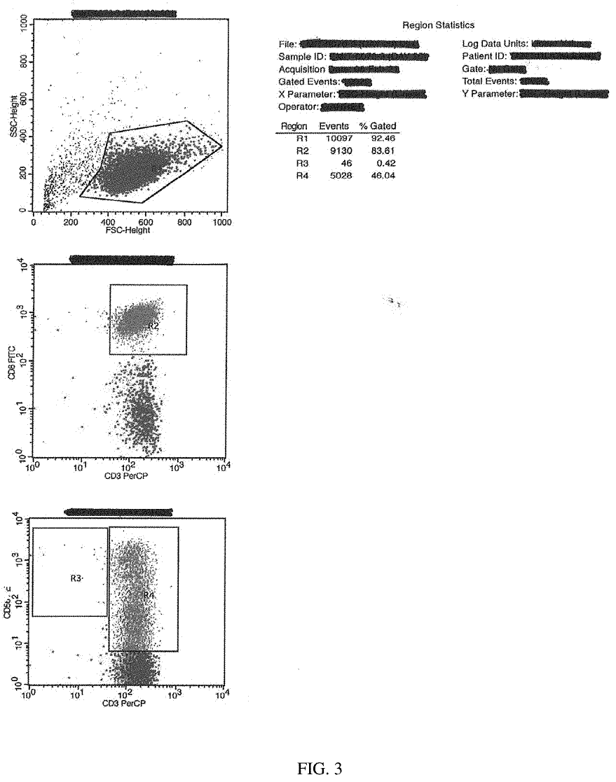

FIG. 3 illustrates the representative composition of CIK cells expanded and generated from the present invention comprising CD3⁺CD8⁺ T-lymphocytes, CD3⁻CD56⁺ NK cells and CD3⁺CD56⁺ NKT cells which was determined using flow cytometry analysis.

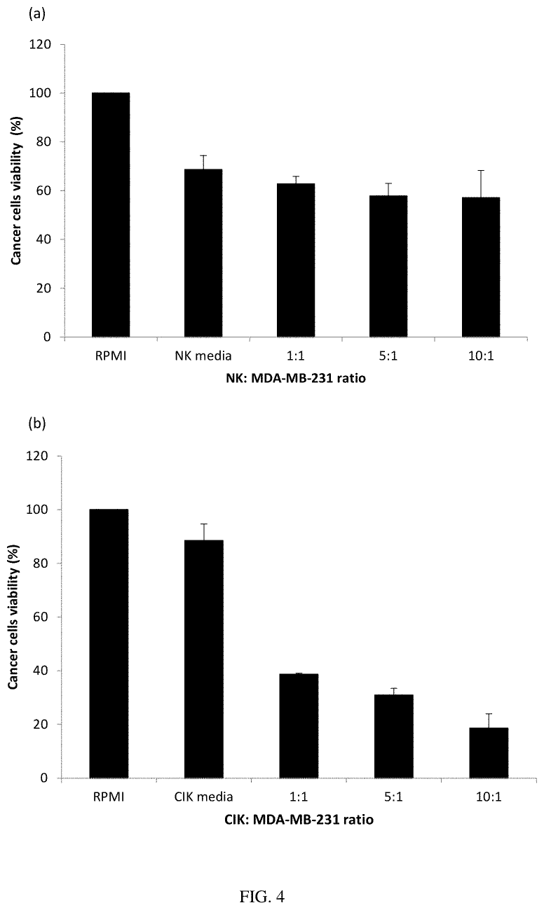

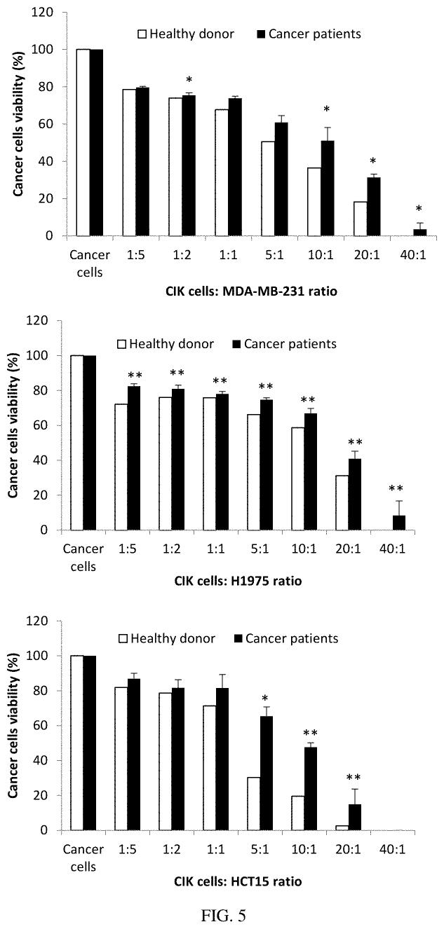

FIG. 4 demonstrates that the expanded and generated population of CIK cells in the healthy donors have greater in vitro cytotoxic killing effects against breast cancer cell line, MDA-MB-231, as compared to NK cells. At E:T ratio of 1:1, CIK cells exhibited approximately 61.3% killing effect against MDA-MB-231 cell line, compared to NK cells which only killed approximately 37.2% of the cells. In addition, the cytotoxicity effect of CIK cells against MDA-MB-231 cells was in a dose-dependent manner. For instance, at a higher E:T ratios of 5:1 and 10:1, only 30.9% and 18.6% of viable cells were observed, respectively. Thus, it is further demonstrated that the CIK cells expanded and generated from the present invention comprising predominantly NKT cells, possess superior cytotoxic effects as compared to NK cells alone.

A study on the tumour killing effect of CIK cells of the present invention in various cancer cell lines was also carried out. 80 mL of peripheral blood was withdrawn from four cancer patients (mean age of 74±11 year-old, male) and a healthy donor (45 year-old, female) using simple venipuncture. The clinical characteristics of the cancer patients and healthy donor were listed in TABLE 2. The samples were isolated and induced in accordance with the present invention. The cytotoxicity of CIK cells were assessed against MDA-MB-231 breast cancer, H1975 lung cancer and HCT15 colorectal cancer cells at E:T ratios of 1:5, 1:2, 1:1, 5:1, 10:1, 20:1 and 40:1 using MTT assay.

TABLE 2
Clinical characteristics of subject during baseline assessments, total CIK
cell count and sub-fraction of T-lymphocytes, NKT cells and NK cells
count during 21 days of culture

| | | | | Cell Count | | T cell count/ | NK Count/ | NKT Count/ |
|---|---|---|---|---|---|---|---|---|
| Subject | | | | (×10⁹ cells) | | CD3+CD8+ | CD3−CD56+ | CD3+CD56+ |
| ID | Age | Gender | Diagnosis | D0 | D21 | (%) | (%) | (%) |
| 1 | 79 | M | Pancreatic cancer | 0.084 | 11.8 | 68.75 | 0.01 | 17.87 |
| 2 | 82 | M | Luing cancer | 0.14 | 9.0 | 51.17 | 36.89 | 10.72 |
| 3 | 78 | M | Colon cancer | 0.06 1 | 6.5 | 85.91 | 0.46 | 27.81 |
| 4 | 57 | M | Prostate cancer | 0.172 | 11.7 | 91 67 | 0.16 | 25.45 |
| 5 | 49 | F | Healthy donor | 0.116 | 8.3 | 87.99 | 0.45 | 19.61 |

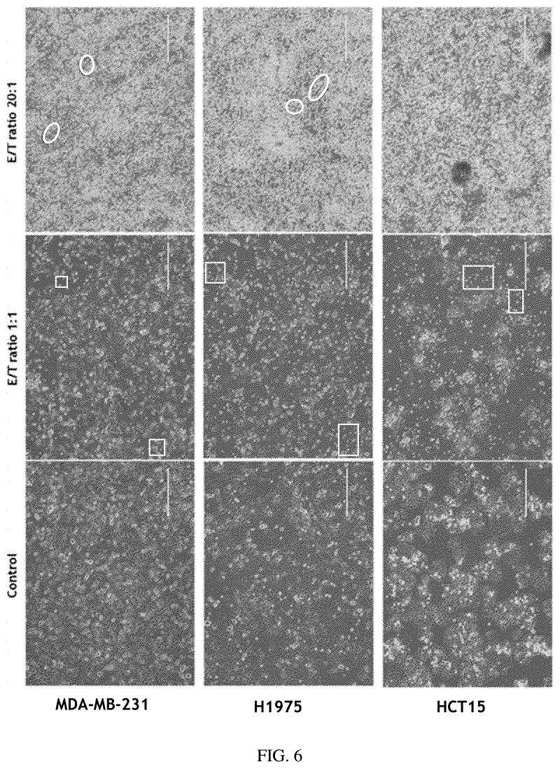

After 14-21 days of culture, the median expansion number in cancer patients was 10.35±2.52×10⁹ cells, which was comparable with the expansion number in the healthy donor (8.3×10⁹ cells). In addition, the median proportion of CD3⁺CD56⁺ NKT cells in cancer patients and healthy donors was also similar, which is 20.66%±7.38% and 19.61%, respectively. FIG. 5 showed that generated CIK cells from the cancer patients demonstrated significant cytotoxic killing activity against all the cancer cells tested in a dose-dependent manner. It was observed that CIK cells generated from both healthy donor and cancer patients showed a comparable pattern of tumour killing effect against breast, lung and colorectal cancer cells. Surprisingly, at the highest E:T ratio of 40:1, CIK cells from healthy donor caused 100% cell death, while CIK cells from cancer patients also showed more than 90% of cytotoxicity effect against all cancer cells. Hence, this demonstrates that the in vitro results reaffirmed the capacity of CIK cells of the present invention to induce cell death effectively in various cancer cells.

Observation of the tumour-killing effect of the CIK cells against cancer cells at different ratios is illustrated in FIG. 6. After 72 hours, naturally occurred cell death was observed in the control wells in all tested cancer cell lines which is common when the cultures become overcrowded and with no fresh culture medium given. In the well treated with CIK cells at 1:1 ratio, the number of cancer cells was relatively lower, as compared to the control well. It was observed that the CIK cells treatment exerted cytotoxic effects against the cancer cells, thus killing and inhibiting the growth of cancer cells. At a higher E:T ratio of 20:1, whereby the concentration of CIK cells was 20× greater than the cancer cells, there were only a small number of cancer cells remaining which adhered to the surface of the culture.

The data showed that high concentration of CIK cells was effective in suppressing and killing most of the cancer cells, thus achieved >90% of cytotoxic killing activity which also suggested that higher number of CIK cells correlates with greater ability to kill and suppress the cancer cells.

Based on the results above, the present invention provides a solution to expand and generate sufficient CIK cells from a relatively small volume of peripheral venous blood, wherein T-lymphocytes have been increased at least 140 times from its original number, with at least 8 billion cells produced. Simultaneously, the cell viability achieved is at least 90% and the percentage of CD3⁺CD56⁺ NKT cells generated is at least 20%.

The population of CIK cells generated from the present invention were shown to possess efficacious cytolytic activity towards cancer cell lines and therefore have a promising potential in cellular immunotherapy for treatment of cancer.

Having described preferred embodiments of the present invention with reference to the accompanying drawings, it is not intended that these embodiments and examples illustrate and describe all possible forms of the present invention, and it is to be understood that the present invention is not limited to those precise embodiments, and that various changes and modifications may be effected therein by a person having ordinary skill in the art without departing from the scope of the present invention as defined in the appended claims.

1. A method of expanding and generating a population of cytokine-induced killer (CIK) cells from peripheral blood mononuclear cells comprising steps of:
a) separating the mononuclear cells from the peripheral blood;
b) transferring the separated mononuclear cells into a culture medium;
c) adding cytokines into the culture medium to induce expansion and generation of the CIK cells; and
d) obtaining the expanded and generated population of CIK cells.

2. The method as claimed in claim 1 wherein the peripheral blood is collected from a subject by way of venipuncture in a volume ranging from 60 mL to 80 mL.

3. The method as claimed in claim 1 wherein the step of separating the mononuclear cells from the peripheral blood is carried out by way of density centrifugation at 800×g for 30 minutes and at room temperature.

4. The method as claimed in claim 1 wherein the culture medium comprises a mixture of T-lymphocyte culture medium, human autologous plasma and interferon gamma (IFN-γ).

5. The method as claimed in claim 4 wherein the IFN-γ is present at a concentration of 1000 U/mL.

6. The method as claimed in claim 4 wherein the human autologous plasma is present at a volume of 0.5%.

7. The method as claimed in claim 1 wherein the culture medium is placed in a tissue culture flask which is coated with an extracellular matrix protein or muromonab CD3 (OKT3) antibody.

8. The method as claimed in claim 1 wherein the extracellular matrix protein includes laminin, collagen, fibronectin or vitronectin.

9. The method as claimed in claim 1 wherein the mononuclear cells are cultured in the culture medium in a duration from 12 to 24 hours.

10. The method as claimed in claim 1 wherein the cytokines include interleukin-2 (IL-2) and interleukin-15 (IL-15).

11. The method as claimed in claim 1 wherein the step to induce expansion and generation of the CIK cells further comprises steps of:
a) adding IL-2 in a concentration ranging from 300 U/mL to 1000 U/mL and OKT3 in a concentration ranging from 100 ng/mL to 400 ng/mL in the culture medium for another duration from 12 to 24 hours;
b) adding IL-2 in a concentration of ranging from 300 U/mL to 1000 U/mL in every 2-3 days for a period of 14-21 days; and
c) adding IL-15 in a concentration ranging from 10 ng/mL to 50 ng/mL on day 4 and day 8 of culture.

12. The method as claimed in claim 1 wherein the step to culture the cells further comprises a step of replacing fresh culture medium in every 2-3 days continuously for a period of 14-21 days.

13. The method as claimed in claim 1 wherein the expanded and generated population of CIK cells are obtained in a form of suspension of cells after 14-21 days of culture.

14. The method as claimed in claim 1 wherein the expanded and generated population of CIK cells includes T-lymphocytes, natural killer (NK) cells and natural killer-T (NKT) cells.

15. The method as claimed in claim 14 wherein the T-lymphocytes are positive for a selected group of surface markers including CD3 and CD8.

16. The method as claimed in claim 14 wherein the NK cells are negative for surface marker CD3 and positive for surface marker CD56.

17. The method as claimed in claim 14 wherein the NKT cells are positive for a selected group of surface markers including CD3 and CD56.

18. Use of the CIK cells as claimed in claim 1 in cellular immunotherapy for treatment of cancer.
